(19) United States
(12) Patent Application Publication
(10) Pub. No.: US 2024/0091073 A1
(43) Pub. Date: Mar. 21, 2024

(54) DISPOSABLE ABSORBENT PANTS WITH ELASTICIZED WAIST PANEL STRUCTURE AND OBSCURING PRINT PATTERNS

(71) Applicant: The Procter & Gamble Company, Cincinnati, OH (US)

(72) Inventors: Sion AGAMI, Mason, OH (US); Tara M. HALE, Cincinnati, OH (US)

(21) Appl. No.: 17/940,626

(22) Filed: Sep. 8, 2022

(51) Int. Cl. A61F 13/49; A61F 13/496; A61F 13/514

(52) U.S. Cl. CPC A61F 13/49011; A61F 13/496; A61F 13/51496; A61F 2013/15243

(57) ABSTRACT

Disclosed is an adult disposable absorbent pant having front and rear panels each comprising stretch laminate material. The stretch laminate material may have an inner layer and an outer layer, each formed of nonwoven web material, and a plurality of longitudinally spaced, laterally extending and laterally pre-strained elastic strands therebetween. The plurality of elastic strands in at least one of the front and rear panels has an average longitudinal strand spacing of 2 mm to 6 mm, and imparts a Rugosity Area to the panel. The Rugosity Area bears a pattern of printing, visible from outside the pant. The pattern of printing may be configured to impart a Percent Rugosity Area with a Color Contrast of at least 50 percent, for the panel. The pattern of printing serves to obscure rugosity of the panel.

### BACKGROUND

In recent years 68 populations in many countries have shifted toward middle-aged and older demographic groups. These demographic groups represent markets with relatively increased demands for products and services addressed to concerns associated with aging.

One such concern is adult urinary incontinence. Urinary incontinence can result from or be exacerbated by a variety of health conditions, and even normal experiences such as childbearing.

Disposable absorbent pants for persons experiencing urinary incontinence have been marketed for a number of years. These products have traditionally had similarities to disposable baby diapers or disposable children's training pants, the main difference being size. One design type is known as the “belted” or “balloon” type pant, which is formed of one or more broad belts or sheets of material that encircle the wearer's waist and lower torso, forming front and rear waist panels, bridged by a structure that connects front and rear belt portions through the wearer's crotch area. The crotch structure includes an absorbent structure designed to receive, contain and store urine until the time the pant is changed. The belt/sheet or waist panel(s) are typically formed of stretch laminate material, formed of two layers of nonwoven web material sandwiching an elastomeric material (in the form of, e.g., laterally-arranged strands or strips, film, etc.) that has been laminated between the nonwoven web layers in a laterally strained (i.e., pre-strained), condition. Upon relaxation of the stretch laminate material following manufacture, the elastomeric material contracts along the lateral direction, causing the sandwiching nonwoven web layers to gather in the lateral direction, forming ruffles of incrementally gathered nonwoven web material.

While the ruffles of gathered material allow the stretch laminate to stretch in the lateral direction during donning and wear by the wearer, unless the laminate is stretched to the full extent of the pre-contraction dimensions of the nonwoven web layers (which would be unusual for normal wear of a pant sized according to conventional sizing practice, by a wearer of intended size), the ruffles impart noticeable rugosity and caliper to the stretch laminate and thus to the front and rear waist panels.

Additionally, disposable absorbent pants typically include an absorbent structure disposed in a crotch region of the pant. The absorbent structure will include added layers of material such as one or more batts of absorbent fibers, deposits or accumulations of particles of absorbent gelling material, added nonwoven layers, etc. These added layers impart added caliper and bulk to the crotch region, and may also impart noticeable discontinuities to the outer contours of the pant as worn, e.g., a noticeable step up in caliper about the peripheral edges of the absorbent structure. Further, when the front and rear waist panels are stretched laterally as during wear (pulling out the gathers to some extent), they may become more translucent, and edges of the absorbent structure may become readily visible from outside.

As a result of these features, the pant product may visually resemble a disposable child's training pant, rather than an ordinary undergarment.

This resemblance has been a source of anxiety and discomfort for some wearers. Such wearers may be unhappy using products that may be associated with aging and loss of control of bodily functions, or be simply unhappy or embarrassed about wearing an undergarment that bears resemblance to conventional disposable children's training pants.

In these circumstances, any improvement to traditional designs and materials for adult incontinence pants, which is efficient for manufacturing while providing an appearance more closely resembling that of an ordinary undergarment, may provide competitive advantages to the manufacturer thereof.

### BRIEF DESCRIPTION OF THE DRAWINGS

FIG. 1 is a simplified perspective view of an example of a pair of disposable absorbent underpants (hereinafter, “underpant”, or “pant”), as if might appear while being worn by a standing wearer (wearer not shown).

FIG. 2A is a schematic plan view of a pant structure with front and rear panels separated at the side/hip regions, as it would appear laid out flat on a horizontal surface, outward-facing surfaces facing up.

FIG. 2B is a schematic longitudinal cross section of the pant structure shown in FIG. 2A, taken through line 2B-2B shown in FIG. 2A.

FIG. 3 is a view of the pant structure of FIG. 2A, depicting circumscribed Rugosity Areas.

FIG. 4 is a view of the pant structure of FIG. 2A, depicting an example of an imprinted rugosity-obscuring print pattern on the front and rear panels.

FIG. 5A is a view of the pant structure of FIG. 2A, illustrating a superimposed Rugosity Grid.

FIG. 5B is a view of another example of a pant structure, illustrating a superimposed Rugosity Grid.

FIG. 6 is a view of the pant structure of FIG. 4, with the superimposed Rugosity Grid as shown in FIG. 5.

FIGS. 7A-7E are views of non-limiting, illustrative examples of potential rugosity-obscuring, relatively regular print patterns.

FIGS. 8A and 8B are magnified views of non-limiting, illustrative examples of potential rugosity-obscuring, relatively less regular or more random print patterns.

FIGS. 9A and 9B are more magnified view of the print patterns of FIGS. 7A and 7B, respectively, illustrating how the patterns provide Color Contrast within square units of a superimposed Rugosity Grid.

### DEFINITIONS

An “adult” disposable absorbent pant is a disposable absorbent underpant product that is marketed or offered for retail sale in association with any combination verbal, graphic or pictorial information indicating, in effect, that the product is configured to be worn by adults, men or women, and/or is configured for purposes of managing adult bladder leaks or adult urinary incontinence. Alternatively, an “adult” disposable absorbent pant is one that is marketed or offered for retail sale without any associated information about the intended wearer or user, beyond a designation of size generally associated with adults. By contrast and by way of illustration, disposable baby diapers and children's disposable absorbent pants are typically if not always marketed or offered for retail sale in association with various combinations of verbal, graphic and/or pictorial information indicating, in effect, that the product is to be applied to and/or worn by infants, children undergoing toilet training, or children, boys or girls experiencing childhood enuresis (bedwetting). Alternatively, an “adult” disposable absorbent pant is one that has a waist size of at least 35 cm.

With respect to a print pattern, a “Color Contrast” is any point, line or path of juxtaposition of a deposit of printed ink of a first color on a substrate, with either an unprinted area of the substrate immediately adjacent thereto, or a printed ink of a second color immediately adjacent thereto, where the unprinted area or the second color contrasts with the first color. A Color Contrast may in many examples be simply visually detected, but where differences between juxtaposed colors or shades thereof are relatively subtle, a “Color Contrast” may be identified via machine assistance as described in the Color Contrast Measurement Method described herein.

A “Rugosity Grid” is an imaginary grid of uniformly-sized, mutually adjacent square units of surface area defined by imaginary longitudinal and lateral lines superimposed over a Rugosity Area as described herein, wherein the lateral lines are evenly longitudinally spaced at two times the average spacing of elastic strands defining the Rugosity Area, and the longitudinal lines, perpendicular to the lateral lines, are evenly laterally spaced, also at two times the average strand spacing. Herein, strand spacing is expressed and to be measured center-to-center.

“Lateral”—with respect to a disposable absorbent pant refers to a direction parallel to a horizontal line tangent to the front surfaces of the upper portions of wearer's legs proximate the torso, when the pant is being worn normally and the wearer has assumed an even, square, normal standing position. A “width” dimension of any component or feature of an article such as a disposable absorbent pant is measured along the lateral direction. When front and rear panels of a disposable absorbent pant are separated at the side/hip regions and the structure is laid out flat on a horizontal planar surface, the “lateral” direction corresponds with the lateral direction relative the structure when it is worn, as defined above. When front and rear panels of a disposable absorbent pant are separated at the side/hip regions and the structure is laid out flat on a horizontal planar surface, “lateral” refers to a direction perpendicular to the longitudinal direction and parallel to the horizontal planar surface. With respect to a disposable absorbent pant, the term “x-direction” is interchangeable with the term “lateral direction.”

The “lateral axis” of a disposable absorbent pant, with the front and rear panels separated at side/hip regions, and the entire structure opened and laid out flat on a horizontal planar surface, is a lateral line lying in an x-y plane and equally dividing the length of the pant structure as laid out. A lateral axis is perpendicular to a longitudinal axis.

“Longitudinal”—with respect to a disposable absorbent pant, refers to a direction perpendicular to the lateral direction. A “length” dimension of feature of the pant or feature thereof is measured along the longitudinal direction. When front and rear panels of a disposable absorbent pant are separated at the side/hip regions and the structure is laid out flat on a horizontal planar surface, the “longitudinal” direction is perpendicular to the lateral direction relative the pant when it is worn, as defined above. With respect to a disposable absorbent pant, the term “y-direction” is interchangeable with the term “longitudinal direction.”

The “longitudinal axis” of a disposable absorbent pant is a longitudinal line lying in an x-y plane and equally dividing the width of the pant, when the front and rear panels of the pant are separated at side/hip regions, and the structure is laid out flat on a horizontal planar surface. A longitudinal axis is perpendicular to a lateral axis.

Herein, the term “rayon” is used generically to include any fiber spun from regenerated cellulose, including but not limited to viscose, lyocell, etc.

“x-y plane,” with reference to a disposable absorbent pant, with front and rear panels separated at side/hip regions and the structure laid out flat on a horizontal planar surface, means any horizontal plane occupied by the horizontal surface occupied by any layer component of the pant. With respect to manufacture or processing of a material web, the term “x-y plane” refers to a plane substantially occupied by a major surface of the material web.

With respect to manufacture or processing of a material web, the term “x-direction” is interchangeable with the term “cross direction.”

With respect to manufacture or processing of a material web, the term “y-direction” is interchangeable with the term “machine direction.”

“z-direction,” with respect to a disposable absorbent pant, when the front and rear waist portions are separated at side/hip regions and the structure is laid out flat on a horizontal planar surface, is a direction perpendicular/orthogonal to the x-y plane. With respect to manufacture or processing of a material web, the term “z-direction” refers to a direction orthogonal to an x-y plane substantially occupied by a major surface of the material web.

The terms “top,” “bottom,” “upper,” “lower,” “over,” “under,” “beneath,” “superadjacent,” “subjacent,” and similar terms relating to relative vertical positioning, when used herein to refer to layers, components or other features of a disposable absorbent pant, are relative the z-direction and are to be interpreted with respect to the pant as it would appear with front and rear panels separated at side/hip regions, with the structure laid out flat on a horizontal planar surface, with its wearer-facing surfaces oriented upward and outward-facing surfaces oriented downward.

With respect to a disposable absorbent pant, or a component thereof, “wearer-facing” is a relative locational term referring to a feature of the component or structure that when in use lies closer to the wearer than another feature of the component or structure. For example, a topsheet has a wearer-facing surface that lies closer to the wearer than the opposite, outward-facing surface of the topsheet.

With respect to a disposable absorbent pant, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Machine Direction” or “MD” as used herein with respect to a disposable absorbent pant refers to a direction parallel to the movement of the article or component through processing/manufacturing equipment.

“Cross Direction” or “CD” as used herein with respect to a disposable absorbent pant refers to a direction perpendicular/orthogonal to the machine direction, and lying along an x-y plane substantially occupied by a major surface of the pant.

“Predominant,” and forms thereof, when used to characterize a quantity of weight, volume, surface area, etc., of a pant or component thereof, constituted by a composition, material, feature, etc., means that a majority of such weight, volume, surface area, etc., of the pant or component thereof is constituted by the composition, material, feature, etc.

Throughout the present description, a material or composite of materials is considered to be “elastic” or “elastomeric” if, when a tensile force is applied to the material along a stretch direction, the material or composite can be extended along the stretch direction to an elongated length of at least 150% of its original relaxed length (i.e. can extend at least 50%), without rupture or breakage which substantially damages the material or composite, and when the force is removed from the material or composite, the material or composite recovers at least 40% of such elongation. In various examples, when the force is removed from an elastically extensible material, the material or composite may recover at least 60% or even at least 80% of its elongation.

The “stretch direction” of a stretch laminate is the direction along which the laminate will most readily undergo elastic stretch and contraction. In a stretch laminate in which one or more elastic members are incorporated into the laminate while in a pre-strained condition, the stretch direction is the direction along which the elastic member(s) are pre-strained. The “trans-stretch direction” of a stretch laminate is the direction perpendicular to the stretch direction.

“Film” means a skin-like or membrane-like layer of material formed of one or more polymers, which does not have a form consisting predominately of a web-like structure of consolidated polymer fibers and/or other fibers.

A “nonwoven” is a manufactured sheet or web of directionally or randomly oriented fibers which are first laid down to form a batt and then consolidated and bonded together by friction, cohesion, adhesion or one or more patterns of bonds and bond impressions created through localized compression and/or application of pressure, heat, ultrasonic or heating energy, or a combination thereof. The term does not include fabrics that are woven, knitted, or stitch-bonded with yarns or filaments. The fibers may be of natural and/or man-made origin and may be staple and/or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yarn). Nonwoven fabrics can be formed by many processes including but not limited to meltblowing, spunbonding, spunmelting, solvent spinning, electrospinning, carding, film fibrillation, melt-film fibrillation, airlaying, dry-laying, wetlaying with staple fibers, and combinations of these processes as known in the art. The basis weight of nonwoven fabrics is usually expressed in grains per square meter (gsm).

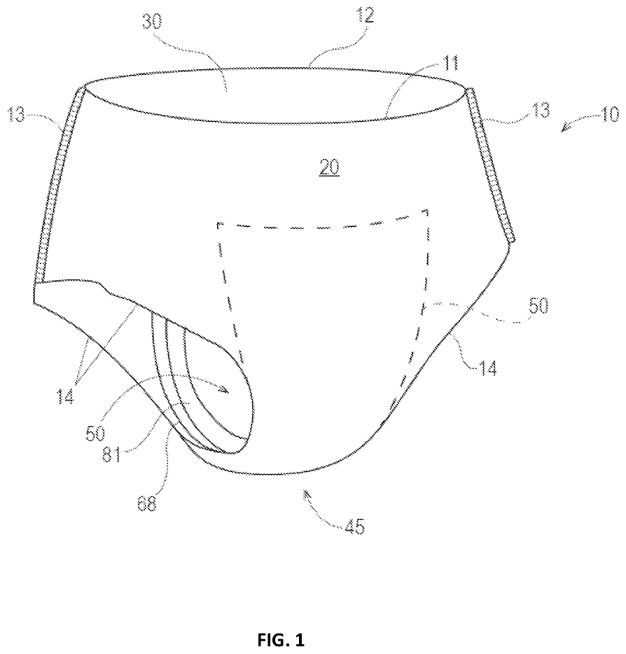

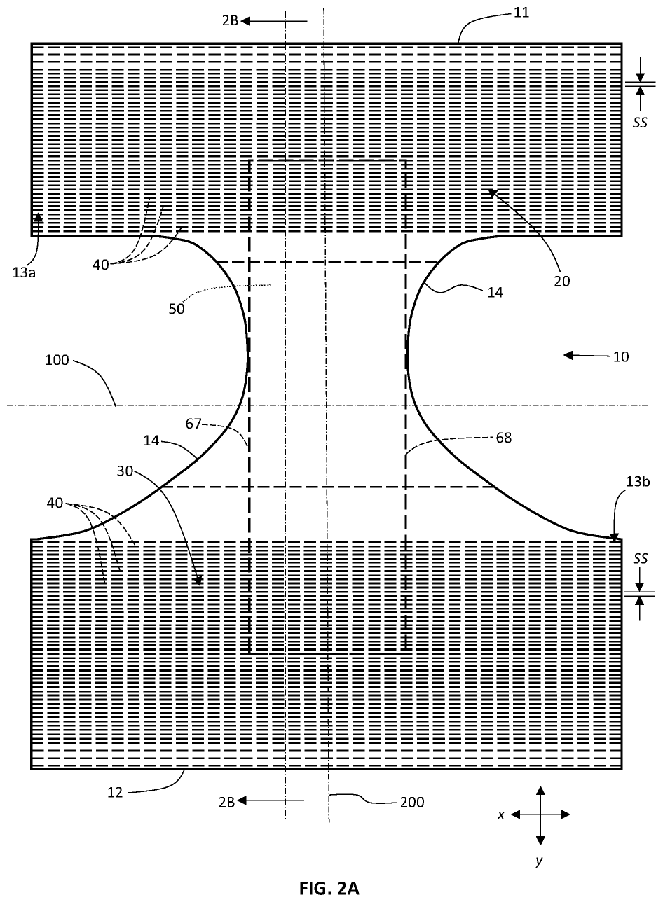

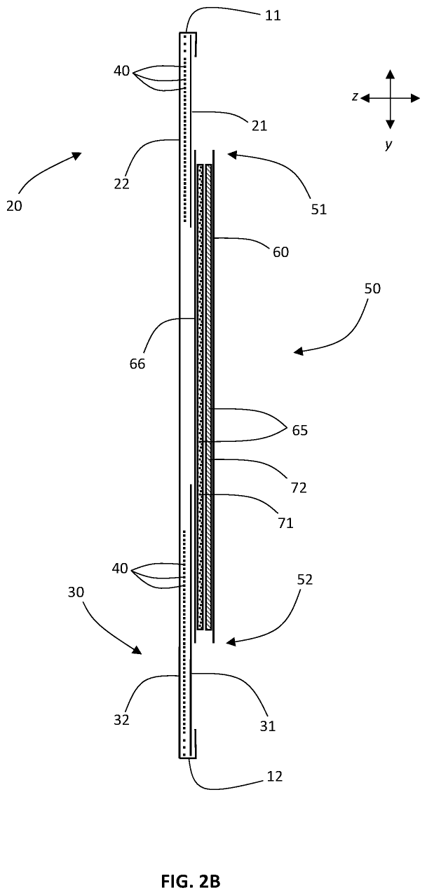

Unless otherwise specified, all dimensions of a pant structure expressed herein (a non-limiting example of which is depicted in FIGS. 1, 2A and 2B) are measured with the structure laid out flat, with non-elastic materials of the structure extended laterally and longitudinally to their full widths and lengths, against any contraction induced by the presence of pre-strained elastic strands or other elastic members.

Wherever a series of successively narrowing ranges of numbers or values for a variable feature are described, it is intended that the description contemplates any combination of the upper and lower limits of the ranges. For example, if a series of ranges is: “from 1 to 10, more preferably from 3 to 8,” it is intended that the description also contemplates “from 1 to 8” and “from 3 to 10.” This is not intended, however, to implicitly alter the meanings or limits of ranges as expressly set forth in the claims.

### DESCRIPTION

FIG. 1 depicts an example of a belt- or balloon-type disposable absorbent pant 10. FIGS. 2A and 2B depict a pant structure shown with front 20 and rear 30 panels separated from side seams 13, with the resulting structure laid out flat and stretched out to the fullest dimensions of the nonwoven web material components, against any contraction induced by included pre-strained elastic members. The pant 10 may have a waist opening defined by a front waist edge 11 and rear waist edge 12 and a pair of leg openings defined by respective leg opening edges 14. Pant 10 may include a belt structure having a front panel 20 and a rear panel 30, which are joined at seam areas 13a, 13b to form side seams 13 and complete the pant structure. Side seams 13 may be butt seams at which seam areas 13a, 13b proximate the side edges of front and rear panels 20, 30 are bonded together by any suitable bonding mechanism. A suitable bonding mechanism may include welding/thermal bonding, in which polymer materials in the front and rear panels 20, 30 are fused together by application of a combination of heat and pressure.

Pant 10 may also include an absorbent pad assembly 50 overlying the front 20 and rear 30 panels to the insides thereof, and bridging them through a crotch region of the pant. Absorbent pad assembly 50 may be bonded to the inside surfaces of front and rear panels 20, 30 by any suitable bonding mechanism, such as a hot melt adhesive applied during the manufacturing process. When laid out flat as depicted in FIG. 2A, the structure may be divided longitudinally in substantially mirror-image halves by a longitudinal axis 200.

Generally, pant 10 may include any features disclosed in U.S. Pat. No. 10,828,208, the entire disclosure of which is incorporated herein by reference to the extent not inconsistent herewith.

Belt/Panel Structure

Referring to FIGS. 2A and 2B, pant 10 may include a belt structure including front panel 20 and rear panel 30. In the example depicted in FIGS. 2A and 2B, outer layers 22, 32 of the front and rear panels are formed of a continuous layer common to both panels, which also wraps about the outside of the pant through the crotch region; this configuration is sometimes called a “unibody” construction. In another possible configuration (not shown), front 20 and rear 30 panels may have discrete, separate outer layers, have no layer in common, and be joined only by the side seams 13 and the absorbent pad assembly. This alternative configuration is sometimes called a “multipiece” construction.

In the example depicted, outer layers 22, 32 of respective front and rear panels 20, 30 may each be formed of a layer of nonwoven web material, which also may serve as the outer layer of the pant through the crotch region. Front and rear panels 20, 30 may also include inner layers 21, 31. Inner layers 21, 31 also may each be formed of layer(s) of nonwoven web material. As suggested in FIG. 2B, in some examples the material forming one or both of outer layers 22, 32 may be wrapped over the respective waist edges 11, 12 and down over the inner layers 21, 31, and adhered thereto by any suitable mechanism (such as adhesive and/or pressure/thermal bonding), to provide waist edges with a neat, finished appearance and feel. In other examples (not specifically shown), the material forming one or both of inner layers 21, 31 may be wrapped over the respective waist edges 11, 12 and down over the outer layers 22, 32, and adhered thereto, for the same purpose.

Suitable nonwoven web materials that may be useful as components to form the inner and outer layers 21, 31, 22, 32 of front and rear panels 20, 30 include, but are not limited to, spunbond, spunlaid, meltblown, spunmelt, solvent-spun, electrospun, carded, film fibrillated, melt-film fibrillated, air-laid, dry-laid, wet-laid staple fibers, and other nonwoven web materials formed in part or in whole of polymer fibers. The nonwoven web materials may be formed predominately of polymeric fibers. In some examples, suitable non-woven fiber materials may include, but are not limited to polymeric materials such as polyolefins, polyesters, polyamide, or specifically, polypropylene (PP), polyethylene (PE), poly-lactic acid (PLA), polyethylene terephthalate (PET) and/or blends thereof. In some examples, the fibers may be formed of PP/PE blends such as described in U.S. Pat. No. 5,266,392. Nonwoven fibers may be formed of, or may include as additives or modifiers, components such as aliphatic polyesters, thermoplastic polysaccharides, or other biopolymers. Further useful nonwovens, fiber compositions, formations of fibers and nonwovens and related methods are described in U.S. Pat. Nos. 6,645,569; 6,863,933 and 7,112,621; and in co-pending U.S. patent application Ser. Nos. 10/338,603; 10/338,610; and Ser. No. 13/005,237.

The individual fibers of the nonwoven web materials may be monocomponent or multicomponent. Multicomponent fibers may be bicomponent fibers, such as in a core-and-sheath or side-by-side arrangement. Often, the individual components comprise polyolefins such as polypropylene or polyethylene, or their copolymers, polyesters, thermoplastic polysaccharides or other biopolymers.

The nonwoven web material may be selected to provide good recovery when external pressure is applied and removed. The nonwoven web material may include a blend of different fibers selected, for example from the types of polymeric fibers described above. In some examples, at least a portion of the fibers may exhibit a spiral curl which has a helical shape. As noted, the fibers may include bicomponent fibers, which are individual fibers each comprising different materials, usually a first and a second polymeric material. It is believed that the use of side-by-side bi-component fibers is beneficial for imparting a spiral curl to the fibers.

In order to enhance tactile and/or visual perceptions of softness of a nonwoven web material, it may be treated by hydrojet impingement, which may also be known as hydroenhancement, hydroentanglement or hydroengorgement. Examples of such nonwoven web materials and processes are described in, for example, U.S. Pat. Nos. 6,632,385 and 6,803,103, and U.S. Pat. App. Pub. No. 2006/0057921.

Other examples of nonwoven web that may be useful may include an SMS web (spunbond-meltblown-spunbond web) made by Avgol Nonwovens LTD, Tel Aviv, Israel, under the designation XL-S70-26; an SSS (spunbond-spunbond-spunbond) web made by Pegas Nonwovens AS in Znojmo, Czech Republic, under the designation 18 XX 01 00 01 00 (where XX=the variable basis weight); an SSS web made by Gulsan Sentetik Dok San VE TIC AS, in Gaziantep, Turkey, under the designation SBXXFOYYY (where XX=the variable basis weight, and YYY=the variable cross direction width); an HESB (hydroenhanced spunbond) web made by First Quality Nonwovens Inc., in Hazelton, Pennsylvania, under the designation SEH2503XXX (where XXX=the variable cross direction width); and a bicomponent SS web.

A nonwoven web material useful as a component to form one or more of layers 21, 31, 22, 32 may be bonded in a pattern of bonds. A batt of loose, e.g., spunlaid, fibers may be passed through the nip between a pair of calender bonding rollers and thereby consolidated and bonded in a pattern of bonds, to add machine-and cross-direction tensile strength and dimensional stability, converting the batt of loose fibers to a coherent and useable nonwoven web material. The bonding may include a pattern of thermal bonds, mechanical bonds, adhesive bonds or a combination thereof, although in some circumstances thermal bonding may be preferred. Thermal bonds may be formed by supplying one or both of the calender rollers or accompanying equipment with a source of heating energy that functions to heat the fibers and cause them to melt and fuse beneath bonding projections in the nip between the calender bonding rollers. One or both of the rollers may be machined, etched or otherwise formed to have a pattern of shaped bonding projections extending radially outward from the cylindrical surface of the roller. When the rollers are maintained in suitably close proximity with their axes in parallel, the batt of fibers passing therebetween will be subjected to pressure concentrated in the nip beneath the bonding projections, and fibers passing through the nip and beneath the bonding projections will be deformed and at least partially fused (by application of heating energy), to form bonds. Each bond will have a shape, and the bonds will have a pattern and spacing, substantially corresponding to the shape, pattern and spacing of the bonding projections on the calender bonding roller.

In some examples, a pattern of thermal bonds used to bond nonwoven web materials used to form one or more of layers 21, 31, 22, 32 may have features described in U.S. Prov. Pat. App. Ser. No. 62/331,650.

Still referring to FIGS. 2A and 2B, layers 21, 31, 22, 32 of either or both of front and rear panels 20, 30 may sandwich one or more elastic members, such as a plurality of laterally extending strands 40 of an elastomeric material, such as an elastane (for example, LYCRA HYFIT fiber, a product of Invista, Wichita, Kansas). Layers 21, 31, 22, 32 may respectively be joined together about elastic strands 40 by adhesive deposited between the layers, by thermal bonds, by compression bonds, or by a combination thereof. In other examples, the one or more elastic members may be strips or a section of film formed of elastomeric material. Elastic strands rather than film, however, may be preferred for the flexibility they provide by enabling individualized setting of prestrain levels, selecting and setting uniform or varying longitudinal spacing therebetween, and preserving a high level of vapor transmission (breathability) through the belt laminate, for purposes of coolness, comfort and skin health. This flexibility helps enable the manufacturer to enhance the fit of the pant structure about the varying contours and sizes of differing wearers' anatomies, and impart a cloth-like appearance to the belt laminate. (For purposes herein, a “strand” is a member having a cross section perpendicular to its longest dimension, the cross section having an aspect ratio of largest dimension to smallest dimension no greater than 2, in an unstrained condition.)

The elastic members can also be formed from various other materials, such as but not limited to, rubbers, styrene ethylbutylene styrene, styrene ethylene propylene styrene, styrene ethylene propylene styrene, styrene butadiene styrene, styrene isoprene styrene, polyolefin elastomers, elastomeric polyurethanes, and other elastomeric materials known in the art, and combinations thereof. In some embodiments, the elastic members can be extruded strand elastics with any number of strands (or filaments).

Elastic strands, if used, may be selected to have a decitex ranging from 50 to 2000, or any integer value for any decitex value in this range, or any range formed by any of these integer values. For purposes herein, however, it may be preferred that elastic strands included to elasticize the major portions of the front and/or rear panels 20, 30 above the bottoms of the side seams 13 have a decitex of from 400 to 1000, more preferably 500 to 900, and still more preferably 600 to 800. In one example, a waistband region of a panel (the region immediately below the waist edge 11 or 12 of the panel) may include from 3 to 12 elastic strands having a higher decitex, and a plurality of strands below the waistband region having a lower decitex. In a more particular example, a waistband region of a pant may include from 3 to 12, more preferably from 4 to 10, and still more preferably from 5 to 10, elastomeric strands having a decitex of from 400 to 1000, more preferably 500 to 900, and still more preferably 600 to 800, and a plurality of strands below the waistband region and above the bottom ends of side seams 13 having a decitex of 300 to 680, more preferably 400 to 580. Use of higher decitex elastomeric strands in a waistband region can be used to provide the pant with relatively greater tension in that region than in lower regions, providing a pant that holds securely and comfortably to the wearer's body about the waistband.

Alternatively, the elastic members may be one or more sections or strips of elastomeric film. Examples of elastomeric films have been described extensively in prior patent applications (see, for example, U.S. Pat. App. Pub. No.

2010/0040826). The film may be created with a variety of resins combined in at least one of several sublayers, the latter providing different benefits to the film. Elastic members may also be in the form of scrim, strips or sections of tape of elastomeric material with their longer dimensions oriented along the stretch direction.

During manufacture of the belt structure, the one or more elastic members such as elastic strands 40, may be pre-strained lengthwise (along the lateral direction) by a desired amount as they are being incorporated into the belt structure. Upon subsequent relaxation of the belt, the one or more elastic members, such as elastic strands 40, will contract toward their unstrained lengths. This causes the sandwiching layers 21, 22 and/or 31, 32 to gather and form ruffles or gathers having ridges and valleys extending generally transversely to the lengths of the elastic strands 40 (i.e., in a longitudinal direction), and also extending in the z-direction. The direction of prestrain corresponds with the stretch direction of the laminate. For purposes herein, and in combination with other features described herein, it may preferred that elastic strands 40 in the front and/or rear panels 20, 30, be pre-strained during manufacture by an amount of from 50% to 290%, more preferably from 90% to 230%, and still more preferably from 120% to 180%, and be affixed between the inner and outer layers of the panels while in such pre-strained condition. (Herein, the amount of prestrain of an elastic strand member is expressed as [((pre-strained unit length)— (unstrained unit length))/(unstrained unit length)]×100%. For example, a unit length of elastic strand pre-strained to twice its unstrained length has a prestrain of 100%.) In combination with one or more of the decitex and strand spacing features described herein, a prestrain level within this range is believed to balance belt structure comfort, close fit, appropriate lateral tension, smoothly distributed over the longitudinal dimension of the belt for causing the absorbent pad assembly to hug the wearer's body, and a cloth-like appearance resulting from the many relatively controlled, small ridges and valleys of ruffles/gathers in the material resulting from prestrain in the elastic strand members.

In the more particular example having waistband region elastic members of differing decitex than those below the waistband region, described above, the waistband region elastic members may be pre-strained during manufacture by an amount of from 110% to 350%, more preferably from 150% to 290%, and still more preferably from 180% to 240%, while the elastic members below the waistband region may be pre-strained during manufacture an amount of from 50% to 290%, more preferably from 90% to 230%, and still more preferably from 120% to 180%.

Where prestrain level for an elastic member is not included in the manufacturer's specifications, it can be calculated, or empirically determined, from known or readily determinable stretch/strain properties of the member and from the level of tensile force introduced into the member as it is incorporated into the belt structure laminate. Alternatively, the amount of prestrain can be measured by making products on the production line with adhesive deposition apparatus turned off for selected samples of the elastic members, and then measuring the stretched and relaxed lengths of the members in the unadhered regions.

The size(s) and shape(s) of the ruffles or gathers may be affected, and may be manipulated, by design of the pattern of joined portions and/or bonding between respective pairs of layers 21, 22 and 31, 32, with respect to each other and with respect to elastic strands 40. The size(s) and shape(s) may also depend upon, and be manipulated by, the selected longitudinal spacing SS of the elastic strands.

As noted, in one example, a stretch laminate may be elasticized by incorporated elastic strands 40 as the elastic stretch mechanism. Elastic strands 40 may have adhesive applied to them prior to lamination (e.g., by a strand coating process), such that, when the web layers 21, 22 and/or 31, 32 are brought together to sandwich the strands, the applied adhesive causes the web layers to be adhered about the strands to form the stretch laminate. The adhesive applied to the elastic strands may be the only adhesive used to hold the laminate together. This configuration helps keep the strands secured between the layers in their longitudinal positions, while allowing the layer materials between the strands to move freely with respect to each other, providing for even formation of gathers/ruffles, and superior breathability. Alternatively, or in addition, adhesive may be deposited upon one or both layers 21, 22 and/or 31, 32 prior to lamination, and may be deposited in a pattern. Examples of methods for applying patterned deposits of adhesive to a nonwoven web substrate to enable manufacture of an elasticized laminate are described in U.S. Pat. No. 8,186,296. In one example, the adhesive pattern selected may be effected by design of a correspondingly designed roller. The pattern of adhesive to be applied may be designed to affect the size(s) and shape(s) of the ruffles or gathers. The layers 21, 22 and/or 31, 32 may be adhesively joined and/or bonded to each other at the locations of adhesive deposits, and remain unjoined or unbonded, or free, of each other at other locations, such that they may move and shift slightly relative each other as the laminate is moved and stretched, as during wear of the article.

Various coating methods and techniques, including strand coating methods and techniques, are shown for example in U.S. Pat. Nos. 5,340,648; 5,501,756; 5,507,909; 6,077,375; 6,200,635; 6,235,137; 6,361,634; 6,561,430; 6,520,237; 6,582,518; 6,610,161; 6,613,146, 6,652,693, 6,719,846 and 6,737,102. The adhesive used may be a hot-melt type adhesive having elasticity and flexibility making it suitable for attaching pre-strained elastic materials to substrates, such as OMNIMELT BLOCKS 22 H2401F, or ZEROCREEP brands such as AVANCE, available from Bostik, Inc., Wauwatosa, Wisconsin.

When bonding of one or both of nonwoven layers 21, 22 and/or 31, 32 is effected using thermal calender bonding, the joining and/or bonding pattern may be designed to affect the size(s) and shapes of the ruffles or gathers. It may be desired in some circumstances that a spunlaid nonwoven web be bonded with a pattern of thermal bonds to a bond area of from 5% to 20%. For purposes described herein it may be desired that bond area be from 8% to 15%. Patterned thermal bonding tends to enhance machine-direction and cross-direction strength and dimensional stability of the resulting bonded nonwoven web, which has benefits in downstream converting and processing operations, and adds tensile strength and robustness to a product in which the web is to form a component. However, thermal bonding also generally increases the stiffness of the resulting bonded nonwoven web. This may have adverse effects on the product consumer's perception of tactile softness of the product surfaces. For example, if the web is used as a layer of a belt structure of a pant product, stiffness imparted to the web may cause the consumer to negatively perceive the belt layer as stiff- or rough-feeling. For this reason, in some circumstances it may be desired to limit bond area to less than 16%, less than 12%, or even less than 10%. Further, imparting certain additional features described in U.S. Prov. Pat. App. Ser. No. 62/331,650 to the bond pattern of a web to be used in a stretch laminate can mitigate the negative effects of stiffening the web, while providing advantages in addition to tensile strength. As disclosed in the above-cited '650 application, nonwoven web material bond patterns having a majority of bonding shapes in the pattern that that are longer in the cross direction than in the machine direction of the nonwoven web may tend to form more controlled, smaller ruffles or gathers, making the belt laminate more cloth-like in appearance.

For purposes of reducing the overall size of the ruffles or gathers formed, and in conjunction with any combination of the features described herein, it may be desired that the average longitudinal spacing SS between subsets of, or all of, the elastic strands 40 above the bottoms of side seams 13 be no greater than 14 mm, more preferably no greater than 10 mm, even more preferably no greater than 7 mm, and still more preferably no greater than 5 mm (Herein, longitudinal spacing between adjacent elastic strands is to be understood to refer to the distance between their axes, not the distance between their nearest outer surfaces.) Through experimentation it has been determined that limiting spacing of elastic strands 40 in this way has the effect of promoting formation of ruffles or gathers that of a controlled small size, thereby providing or enhancing a cloth-like appearance in the stretch laminate.

Rugosity-Obscuring Print

Belt and/or panel structures specifically contemplated herein include those provided with pre-strained, laterally-extending, longitudinally-spaced elastic strands 40, as the mechanism by which the belt/panel structure is imparted with lateral elastic extensibility. A relatively premium product is contemplated, wherein the longitudinal spacing SS of the strands 40 is, on average, 2 mm to 6 mm (Herein, the average strand spacing is the average all of the individual distances by which the laterally-oriented elastic strands in the entirety of the subject belt or panel are spaced, center-to-center.) When average strand spacing is greater than 6 mm, it is believed that consumers will not perceive the product as premium due to the sizes of the gathers and ruffles imparting rugosity the belt/panel. When average strand spacing is less than 2 mm, the gathers and ruffles imparting rugosity become quite small and the belt/panel structure takes on a relatively more premium, cloth-like appearance; however, it will be recognized that practical technical challenges are presented and costs are increased when such small spacing is sought. Accordingly, the print pattern features described herein operate to impart or enhance a cloth-like, premium appearance in belt/panel structures having average longitudinal average strand spacing of 2 mm to 6 mm.

The ruffles of gathered nonwoven material imparted to a stretch laminate, by the inclusion and presence of contracted/relaxed, pre-strained elastic strands 40, cause a pattern of lighter areas and shadowed areas to be visible to the eye, thereby rendering the increased caliper and rugosity of the stretch laminate to be readily perceivable. Generally, the lighter areas appear on portions of the gathers/ruffles that protrude outwardly in the z-direction, and the shadowed areas appear on portions of the gathers/ruffles that are recessed inwardly (toward the wearer) in the z-direction.

It has been discovered, however, that a suitable print pattern may be applied to one or more of the surfaces of the component nonwoven web material components (such as inner and outer layers 21, 31, 22, 32) of a belt/panel structure. It has been discovered that, if the print pattern is suitably “busy” in appearance, i.e., has features that present a suitably visually dense pattern of interfaces or juxtapositions of contrasting colors to the viewer, the pattern of lighter areas and shadowed areas and rugosity imparted by the nonwoven web material gathers becomes less noticeable and/or less obvious to the viewer. It has been learned that such a rugosity-obscuring print pattern may be particularly well-received by adult wearers (differentiated from children and infants), because they may be more aware and conscious about the appearance of the pant when worn. Accordingly, in some examples applicability of the print pattern features described herein may be particularly applicable to disposable absorbent pants having a pant waist size equal to or greater than 35 cm (which can distinguish an adult product from an infant or child product).

As may be appreciated, the size of the ruffles in the belt/panel structure is related in part to the size of the spacing between the elastic strands 40. It has been discovered that a pattern of printing applied to one or more surfaces of the nonwoven web components will be effective for obscuring ruffles if the print pattern presents a Color Contrast within units of an imaginary Rugosity Grid as described and defined herein, to a minimum percentage of a Rugosity Area as defined herein.

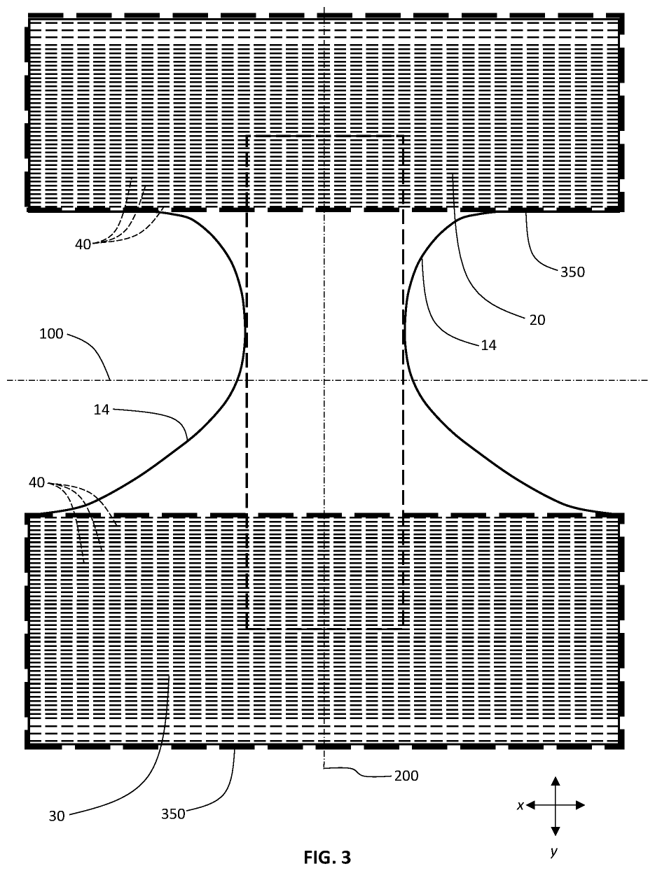

Referring to FIGS. 2A and 3, a Rugosity Area 300 (shown in FIG. 3 circumscribed by a heavy dashed line) for purposes herein is that area of a front or rear panel 20, 30 that is tangent to but entirely above the leg opening edges 14 (when the pant is in assembled condition as in FIG. 1). Rugosity in Rugosity Area 300 is imparted to the stretch laminate of the panels, by contraction of elastic strands 40 that are disposed within the Rugosity Area, causing the nonwoven material(s) of the stretch laminate to laterally gather and ruffle.

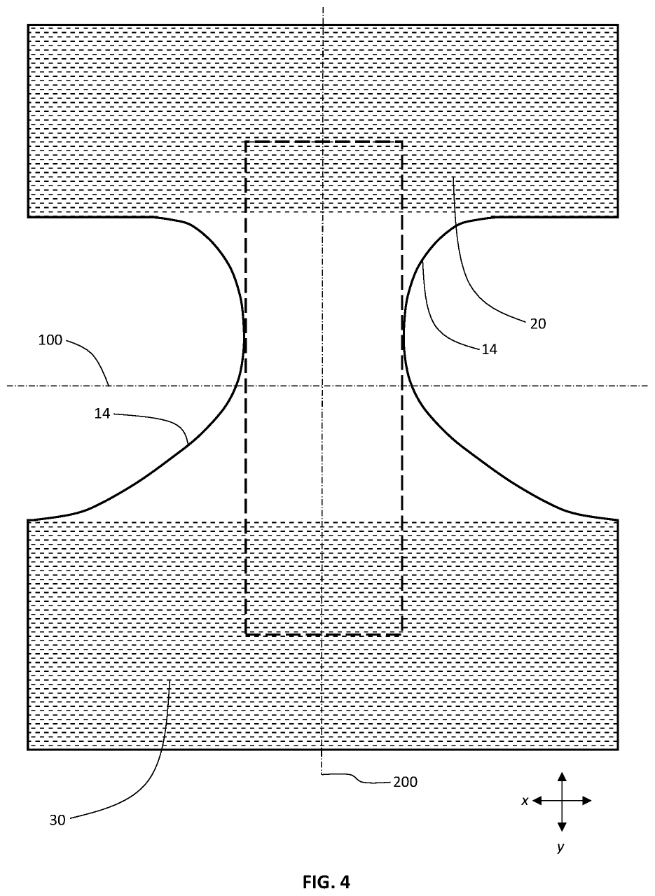

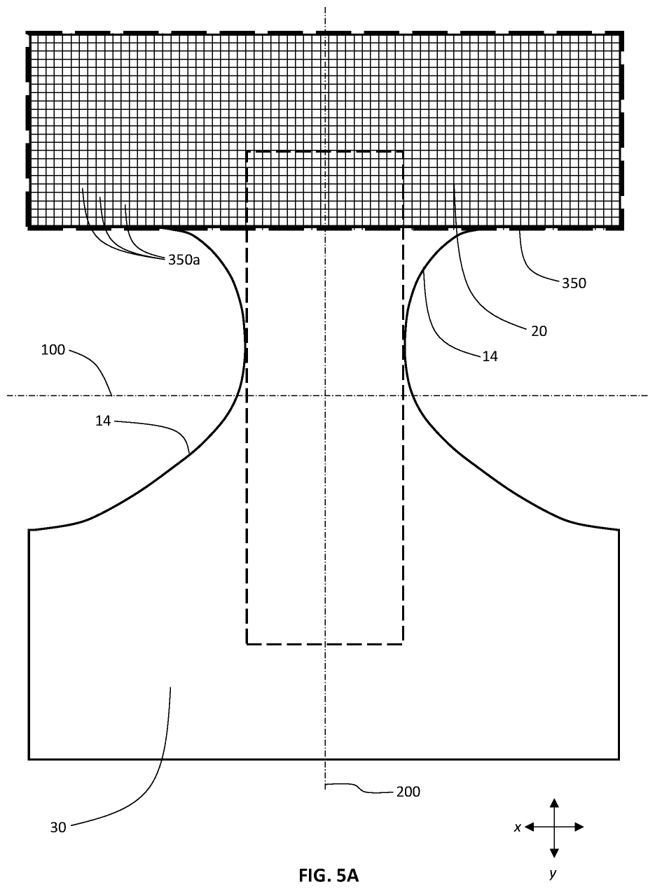

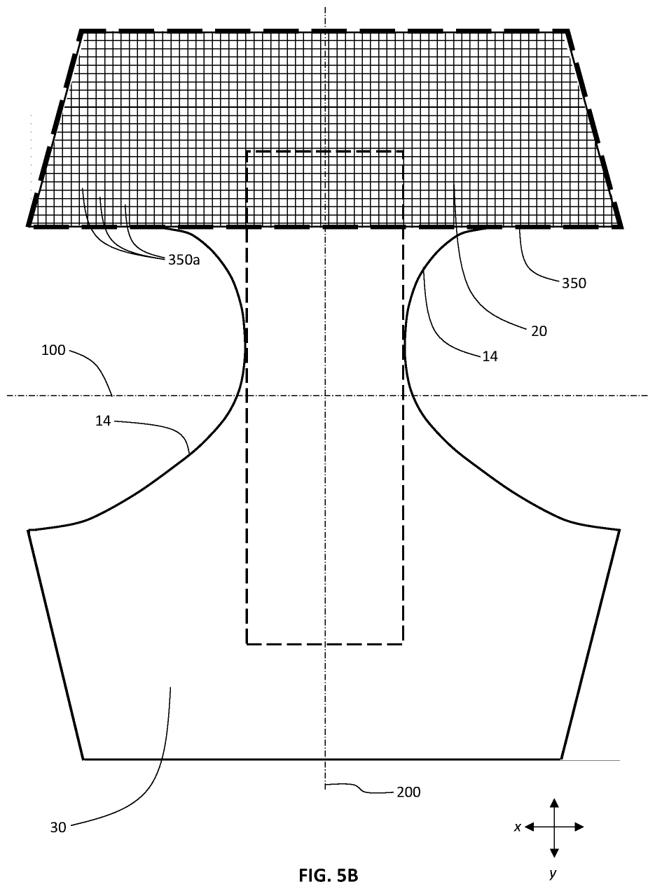

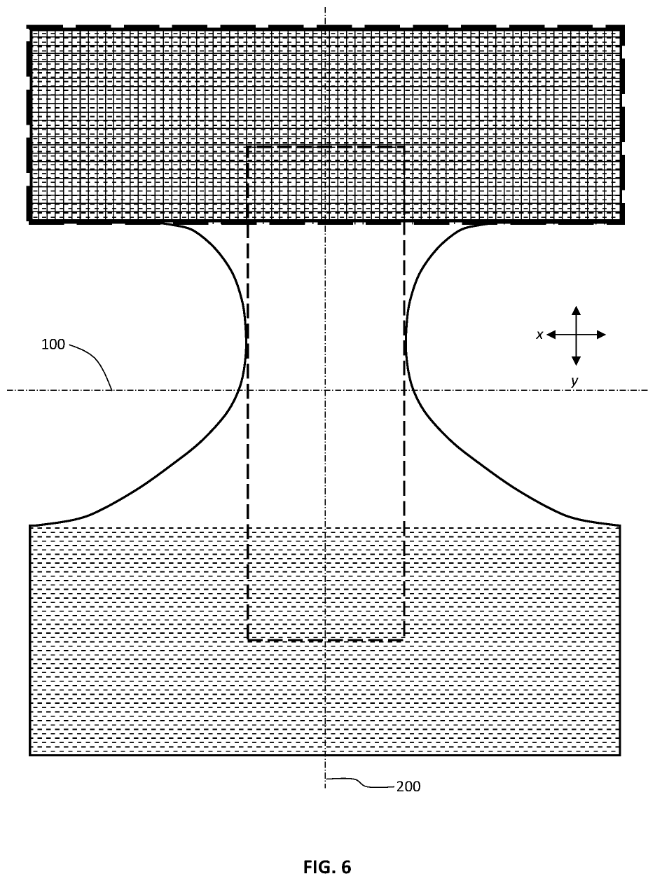

FIG. 4 illustrates one non-limiting example of a suitable print pattern applied to a nonwoven web layer of one or both of front and rear panels 20, 30. The print pattern illustrated includes a pattern of printed, laterally-oriented rod shapes arranged in a staggered pattern. FIG. 5A illustrates an example of the pant with a Rugosity Grid 350 superimposed over the front panel. The Rugosity Grid consists of a group of adjacent/contiguous, identically-sized square units of area 350a within the Rugosity Area, each unit 350a having a length and width dimension equal to 2 times the average longitudinal spacing of the elastic strands 40. FIG. 6 illustrates an example of the pant with a Rugosity Grid 350 superimposed over the front panel, which also bears the print pattern shown in FIG. 4.

FIG. 5B illustrates a Rugosity Grid 350 superimposed over another example of a Rugosity Area 300 of a pant structure, wherein the front and rear panels 20, 30 and correspondingly the Rugosity Areas do not have rectangular profiles. It will be appreciated from the description herein that the number of square units of area 350a that are present within a Rugosity Area 300 will become relevant for purposes herein. For purposes of clarity, in order to count square units of area 350a within a Rugosity Area 300, only whole units (i.e., units that are entirely present within the boundary of the Rugosity Area 350) are counted; partial units within the boundary are not to be counted. Along the lateral direction, the Rugosity Grid is to be disposed symmetrically about longitudinal axis 200. Along the longitudinal direction, the Rugosity Grid is to be located with respect to the lower boundary (the portion of the boundary nearest the lateral axis 100) of the Rugosity Area 300 such that whole square units of area 350a are immediately adjacent such lower boundary.

It may be seen in FIG. 6 that at least a portion of one of the rod shapes of the print pattern appears in each of the square units of area 350a of a superimposed Rugosity Grid 350. Provided that the rod shapes are printed in a color that, when viewed from the outward-facing side of the pant, contrasts with the background color of the nonwoven web material, the pattern as illustrated embodies the features that will obscure ruffles/gathers of material, according to the theory described above.

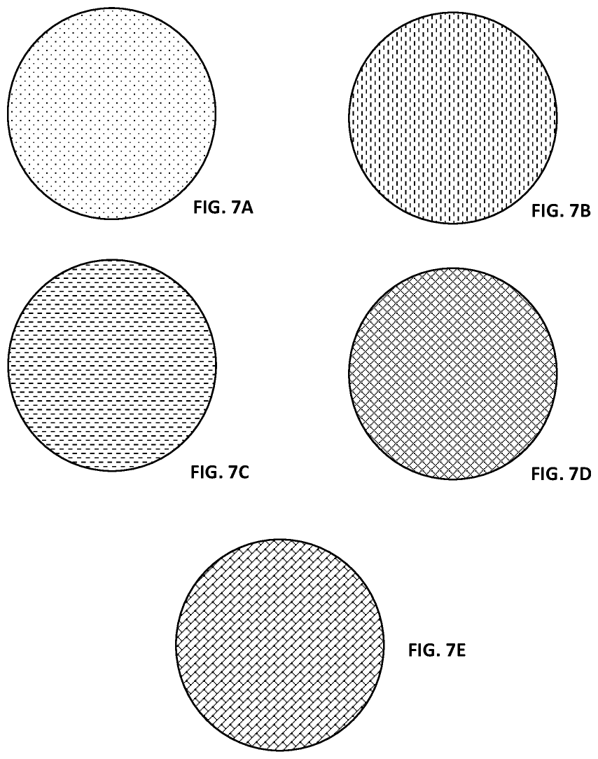

FIGS. 7A-7E illustrate additional non-limiting examples of regular print patterns that may be configured with suitable visual density. Provided they have suitable dimensions such that a Color Contrast as described herein is present within the minimum number of square units 350a of a Rugosity Grid 350 as described herein, these patterns imprinted on a nonwoven web component on a front or rear panel 20, 30 can serve the rugosity-obscuring function described herein.

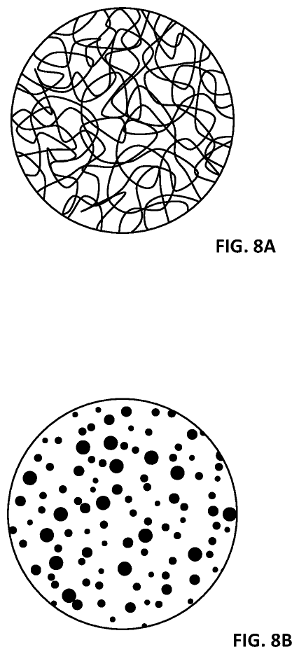

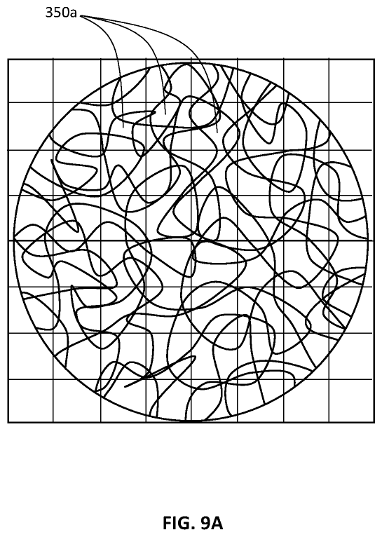

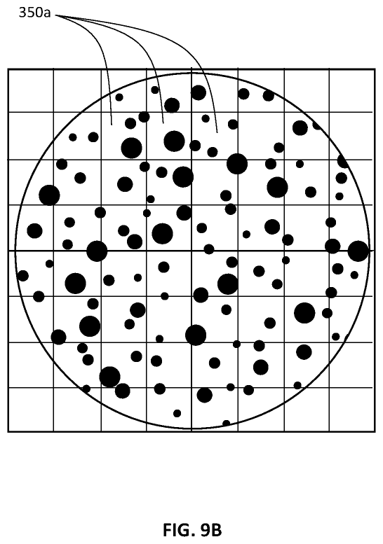

FIGS. 8A and 8B illustrate additional non-limiting examples of irregular print patterns that may be configured with suitable visual density. Provided they have suitable dimensions such that a Color Contrast as described herein is present within the minimum number of square units 350a of a Rugosity Grid 350 as described herein, these patterns imprinted on a nonwoven web component on a front or rear panel 20, 30 can serve the rugosity-obscuring function described herein. FIGS. 9A and 9B illustrate how these irregular patterns can have print elements present that impart Color Contrast within square units 350a of a Rugosity Grid 350.

It may be appreciated that a product designer may prefer not to obscure rugosity of one or more portions of a front and/or rear panel 20, 30 in limited areas, for designs that might intentionally incorporate visible rugosity as an aesthetic feature. Accordingly, for purposes herein, an applied print pattern is deemed sufficiently rugosity-obscuring if it imparts a Color Contrast within at least 50 percent, more preferably at least 65 percent, and even more preferably at least 75 percent, of the square units 350a of a Rugosity Grid 350, superimposed over a Rugosity Area 300 of one or both of front and rear panels 20, 30. This value is expressed herein as Percent Rugosity Area with a Color Contrast, and is measured as described in the Color Contrast Measurement Method described below.

The printing may be applied to one or more of the wearer-facing and outward-facing surfaces of one or both the inner layer and outer layer of the panel having the Rugosity Area. The printing may be applied via any known method for printing on nonwoven web materials.

The Color Contrast described herein is contemplated to be imparted via printing, and not via aperturing, embossing or other deformation of the nonwoven web material within the Rugosity Area.

Absorbent Pad Assembly

Referring to FIG. 2B, absorbent pad assembly 50 may include any combination of components found in disposable diapers and absorbent pants, including but not limited to a liquid impermeable backsheet, a liquid permeable topsheet, an absorbent core structure disposed between the topsheet and backsheet, and elasticized barrier cuffs. Examples and descriptions of components and configurations of such an absorbent pad assembly or central chassis may be found in U.S. Pat. App. Ser. No. 1 3/7 64,945, wherein the chassis described includes components and features that may be included. As described herein, additional features and combinations thereof may be included as well.

Referring to FIGS. 2A and 2B, absorbent pad assembly 50 has a front end 51, rear end 52, and left and right edges 67, 68. Assembly 50 may include a liquid permeable topsheet 60, a liquid impermeable backsheet 66, and an absorbent core structure 65 disposed between the topsheet and backsheet. The topsheet 60 may be formed of a nonwoven web material suitably selected to contain the components of the absorbent core structure while permitting urine to freely pass therethrough, from the wearer-facing surface to the absorbent core structure 65. The backsheet 66 may include or be formed at least in part of a polymeric film material suitably selected to contain the components of the absorbent core structure, and also to contain and prevent passage of urine from the absorbent core structure therethrough, to the outward-facing surface, under ordinary conditions of use. In some examples the backsheet 66 may also include an outer layer formed of a nonwoven web material to provide added strength and impart a more cloth-like feel. In some examples the backsheet film may be formed so as to be breathable, such that it can permit water vapor to pass therethrough, while still preventing aqueous liquid (urine) from passing therethrough, which can help improve comfort of the pant for the wearer. Materials for suitable topsheet and backsheet materials are well-known in the art. The materials of the topsheet and backsheet may be joined and bonded together about their peripheries, to form an envelope structure containing the absorbent core structure 65, by any suitable bonding mechanism, for example, hot melt adhesive.

The absorbent pad assembly 50 may also include a pair of longitudinal barrier cuffs (not shown). Barrier cuffs and associated longitudinal edge structures and elastic members may also be formed of materials and configured as described in any of, for example, U.S. Pat. No. 8,939,957; US2016/270978; US2016/270971; US2016/270980; US2016/270985; US2016/270983; US2016/270979; US2016/270975; US2016/270981, and US2016/270973.

Absorbent Core Structure

The absorbent core structure 65 may include one or more layers that serve differing liquid-handling and storage functions. In the example depicted in FIG. 2B, absorbent core structure 65 may include an absorbent layer 71 and an acquisition layer 72. Absorbent layer 71 may be formed of an absorbent material that tends to attract and retain aqueous liquid such as urine. In some examples, absorbent layer 71 may include a distribution of particles of absorbent gelling material (AGM), also known as superabsorbent polymer (SAP). In some examples, absorbent layer 71 may include a blend of particles of SAP and cellulose fibers such as pulp fibers.

In some examples, the absorbent layer includes SAP particles physically blended with cellulose fibers. “Cellulose” as used herein includes cellulose pulp fibers as well as comminuted wood pulp in the form of fibers, sometimes also referred in the art as “air-felt”. In some examples, the absorbent layer includes more than 70%, or more than 80%, or more than 90%, or more than 95% or even 100% by weight of superabsorbent polymer particles. In some other examples, the absorbent layer includes superabsorbent polymer particles and less than 5% by weight of cellulose, or less than 2% by weight of cellulose, or even substantially no cellulose. In examples wherein the absorbent layer is cellulose free, the only absorbent material in the absorbent layer is the superabsorbent polymer (particles, fibers, etc.). The resulting absorbent core structures have a reduced thickness in the dry state compared to conventional absorbent core structure including cellulose fibers. The reduced thickness reduces overall bulk of the pant and helps to improve the fit and comfort of the pant for the wearer.

The superabsorbent polymer particles may be immobilized on a substrate layer by, for example, a thermoplastic adhesive material.

“Superabsorbent polymer” (or “SAP”) as used herein refers to absorbent materials which are cross-linked polymeric materials that can absorb at least 10 times their weight of an aqueous 0.9% saline solution as measured using the Centrifuge Retention Capacity (CRC) test (EDANA method WSP 241.2-05E).

The SAP used may in particular have a CRC value of more than 20 g/g, or more than 24 g/g, or of from 20 to 50 g/g, or from 20 to 40 g/g, or from 24 to 30 g/g. The SAP useful in the present invention include a variety of water-insoluble, but water-swellable polymers capable of absorbing large quantities of fluids.

The superabsorbent polymer can be in particulate form so as to be flowable in the dry state. Typical particulate superabsorbent polymer materials are made of poly(meth)acrylic acid polymers. However, e.g. starch-based particulate superabsorbent polymer material may also be used, as well polyacrylamide copolymer, ethylene maleic anhydride copolymer, cross-linked carboxymethylcellulose, polyvinyl alcohol copolymers, cross-linked polyethylene oxide, and starch grafted copolymer of polyacrylonitrile. The superabsorbent polymer may be polyacrylates and polyacrylic acid polymers that are internally and/or surface cross-linked. Suitable materials are described in, for example, PCT Patent Applications Nos. WO07/047598, WO07/046052, WO2009/155265 and WO2009/155264. In some embodiments, suitable superabsorbent polymer particles may be obtained by current state of the art production processes as is more particularly as described in WO2006/083584. The superabsorbent polymers are preferably internally cross-linked, i.e., the polymerization is carried out in the presence of compounds having two or more polymerizable groups which can be free-radically copolymerized into the polymer network. Useful crosslinkers include for example ethylene glycol dimethacrylate, diethylene glycol diacrylate, allyl methacrylate, trimethylolpropane triacrylate, triallylamine, tetraallyloxyethane as described in EP0530438, di- and triacrylates, as described in EP0547847, EP0559476, EP0632068, WO93/21237, WO03/104299, WO03/104300, WO03/104301 and in DE10331450, mixed acrylates which, as well as acrylate groups, include further ethylenically unsaturated groups, as described in DE10331456 and DE10355401, or crosslinker mixtures as described for example in DE19543368, DE19646484, WO90/15830 and WO02/32962 as well as cross-linkers described in WO2009/155265. The superabsorbent polymer particles may be externally surface cross-linked, or post cross-linked). Useful post-crosslinkers include compounds including two or more groups capable of forming covalent bonds with the carboxylate groups of the polymers. Useful compounds include for example alkoxysilyl compounds, polyaziridines, polyamines, polyamidoamines, di- or polyglycidyl compounds as described in EP0083022, EP0543303 and EP0937736, polyhydric alcohols as described in DE-C3314019, cyclic carbonates as described in DE-A4020780, 2 oxazolidone and its derivatives, such as N-(2-hydroxyethyl)-2-oxazolidone as described in DE-A19807502, bis- and poly-2-oxazolidones as described in DE-A19807992, 2-oxotetrahydro-1,3-oxazine and its derivatives as described in DE-A19854573, N-acyl-2-oxazolidones as described in DE-A19854574, cyclic ureas as described in DE-A10204937, bicyclic amide acetals as described in DE-A10334584, oxetane and cyclic ureas as described in EP1199327 and morpholine-2,3-dione and its derivatives as described in WO03/031482.

The SAP may be formed from polyacrylic acid/polyacrylate polymers, for example having a neutralization degree of from 60% to 90%, or about 75%, having for example sodium counter ions. Suitable SAP may also for example be obtained from inverse phase suspension polymerizations as described in U.S. Pat. Nos. 4,340,706 and 5,849,816 or from spray- or other gas-phase dispersion polymerizations as described in US2009/0192035, US2009/0258994 and US2010/0068520. In some embodiments, suitable SAP may be obtained by current state of the art production processes as is more particularly described from page 12, line 23 to page 20, line 27 of WO2006/083584.

The absorbent layer 71 may include only one type of SAP, but it may also include a blend of differing types or compositions of SAPs. The fluid permeability of a superabsorbent polymer can be quantified using its Urine Permeability Measurement (UPM) value, as measured in the test disclosed European patent application number EP12174117.7. The UPM of the SAP may for example be of at least 10 x10-7 cm3.sec/g, or at least 30 x10-7 cm3.sec/g, or at least 50 x10-7 cm3.sec/g, or more, e.g. at least 80 or 100 x10-7 cm3.sec/g. The flow characteristics can also be adjusted by varying the quantity and distribution of the SAP used in the absorbent core.

The superabsorbent polymer particles may be spherical, spherical-like, ellipsoid, or irregularly shaped, such as ovoid-shaped particles of the kind that may be obtained from inverse phase suspension polymerizations. The particles may, optionally, be agglomerated at least to some extent to form larger irregular agglomerations of particles.

In some examples, the absorbent layer may be substantially cellulose-free. Airfelt and other cellulose fiber have been used as absorbent fillers in absorbent cores of disposable diapers. Such fiber also has absorbent properties and imparts some absorption capacity to an absorbent layer, but also may be included to provide a structural matrix to hold dispersed particles of superabsorbent polymer particles. While inclusion of such particles enhances absorption capacity, keeping such particles suitably dispersed may be important to prevent the particles from “gel-blocking” in use as they swell with absorbed liquid and block the passageways therebetween which allow liquid to move through deposits thereof, compromising absorption capacity. The inclusion of airfelt or other cellulose fiber as a matrix for superabsorbent polymer particles can serve to reduce or prevent gel-blocking. However, it also imparts bulk to an absorbent layer, even before absorption of any liquids. To reduce the overall size and/or thickness of the absorbent layer, and thereby improve wearer comfort and reduce the bulkiness of the pant for purposes of packaging and shipping volume efficiency, it may be desired to construct an absorbent core using the lowest volumes of core materials possible within performance constraints. Toward this end, examples of suitable materials and constructions for a suitable absorbent core structure are described in, but are not limited to, U.S. patent application Ser. Nos. 12/141,122; 12/141,124; 12/141,126; 12/141,128; 12/141,130; 12/141,132; 12/141,134; 12/141,141;

12/141,143; and Ser. No. 12/141,146; and WO2008/155699. Generally, these applications describe absorbent layer constructions that minimize or eliminate the need for and inclusion of airfelt or other forms of cellulose fiber in combination with particles of superabsorbent polymer particles (“substantially cellulose-free” structures). Suitable methods for forming deposits of superabsorbent polymer particles are additionally disclosed in, for example, EP1621167A2, EP1913914A2 and EP2238953A2.

The superabsorbent polymer particles may be distributed and immobilized on the substrate layer Immobilization may be achieved by applying a thermoplastic adhesive material, which holds and immobilizes the superabsorbent polymer particles, and cellulose when present, on the substrate layer. Some thermoplastic adhesive material may also penetrate into the layer of superabsorbent polymer particles and into the substrate layer to provide further immobilization and affixation. The thermoplastic adhesive material may not only help in immobilizing the superabsorbent polymer particles on the substrate layer but also may help in maintaining the integrity of any included channels (described further below). The thermoplastic adhesive material can help prevent a significant quantity of superabsorbent polymer particles from migrating into the channels.

Thermoplastic adhesive materials suitable for use in the present disclosure includes hot melt adhesives including at least a thermoplastic polymer in combination with a plasticizer and other thermoplastic diluents such as tackifying resins and additives such as antioxidants. Example suitable hot melt adhesive materials are described in EP1447067 A2.

The absorbent layer, absorbent core structure and/or an optional configuration of channels therein may also have any features described in U.S. Pat. App. Pub. Nos. US2014/0163511; US2014/0163503; US2014/0163501; US2014/0163500; US2012/0316526; US2012/0316528; US2014/0163501; and US2014/0371701; and U.S. patent application Ser. No. 14/598,783.

In some examples, the absorbent core structure 65 may include an acquisition layer 72, disposed between the topsheet and the wearer-facing side of the absorbent layer 71. The acquisition layer 72 may be formed of one or more materials providing an open, highly porous structure configured to disperse and dissipate mechanical energy in a flow of urine, while providing interstitial spaces within the structure to serve as a temporary reservoir for the urine until the absorbent layer 71 can capture and retain (absorb) it. The acquisition layer 72 may consist of a single layer or multiple sublayers, such as an upper acquisition sublayer closest wearer's skin and a lower acquisition sublayer disposed between the upper acquisition layer and the absorbent layer 71. The acquisition layer 72 may be disposed so as to be in direct contact with the absorbent layer. Where channels are present in the absorbent layer 71, materials forming the acquisition layer 72 may extend into or fill in the channels or portions thereof; this may be preferred in some circumstances to prevent rapid, unrestricted flow of unabsorbed urine through the channels, which could increase chances of leakage. In some examples, the acquisition layer, or a sublayer thereof, may be bonded to the substrate layer which defines the channels, thus providing a matching surface profile to the acquisition layer.

Acquisition layer 72 may have the form of, e.g., a layer, mat or other body formed of or including, e.g., comminuted cellulose fibers, or other hydrophilic natural, semi-synthetic or synthetic fibers or other material that may be used to form a mat, layer or other body.

In one example, one or both of upper and lower acquisition sublayers may include a non-woven mat of fibers, which may be hydrophilic. Further, according to a certain example, one or both of the upper and lower acquisition layers may include the chemically cross-linked cellulosic fibers, which may or may not form part of a nonwoven material. According to an example, the upper acquisition layer may include a nonwoven, without the cross-linked cellulosic fibers, and the lower acquisition layer may include the chemically cross-linked cellulosic fibers. Further, according to an example, the lower acquisition layer may include the chemically cross-linked cellulosic fibers mixed with other fibers such as natural or synthetic polymeric fibers. According to example examples, such other natural or synthetic polymeric fibers may include high surface area fibers, thermoplastic binding fibers, polyethylene fibers, polypropylene fibers, PET fibers, rayon fibers, lyocell fibers, eucalyptus fibers and mixtures thereof. Suitable non-woven materials for the upper and lower acquisition layers include, but are not limited to SMS material, including a spunbonded, a melt-blown and a further spunbonded layer. In certain examples, permanently hydrophilic nonwovens, and in particular, nonwovens with durably hydrophilic coatings are desirable. Another suitable example includes an SMMS-structure. In certain examples, the nonwovens are porous.

Color Contrast Measurement Method

The Color Contrast Measurement Method is used to measure the magnitude of color difference, AE, within regions of interest (ROI) on a pattern printed on the outward facing surface (i.e. clothing facing surface) of an absorbent article, and to determine the percentage of square units of surface area of a Rugosity Grid, superimposed over a front or rear waist panel, that are occupied by a Color Contrast. A color difference (ΔE) equal to or greater than the minimum amount specified below defines a Color Contrast for purposes herein.

It will be appreciated that color differences indicated by relatively larger ΔE values will be readily visible to the human eye (assuming normal vision without substantial “color blindness”), making machine-assisted determination of a ΔE value superfluous or unnecessary in some examples. The method herein includes description of machine-assisted determination of ΔE values for purposes of identifying a Color Contrast in examples in which color difference(s) are more subtle, i.e., exhibit relatively lower ΔE values approaching the minimum value set forth herein.

The distance between any two colors in 3D space is referred to as the ΔE value. The ROI can be printed regions that contain differing colors, differing intensities, shades or hues of colors, as well as nonprinted adjacent, abutting or surrounding regions. For this method, the size of each ROI is defined by a superimposed Rugosity Grid, as defined herein. A flatbed scanner capable of scanning a minimum of 24 bit color at 72 dpi with manual control of color management (a suitable scanner is an Epson Perfection V850 Pro from Epson America Inc., Long Beach CA, or equivalent) is used to acquire images of the front and rear panels of the absorbent article. The scanner is interfaced with a computer running color calibration software capable of calibrating the scanner against a color reflection IT8 target utilizing a corresponding reference file compliant with ANSI method IT8.7/2-1993 (suitable color calibration software is it Profiler available from X-Rite Grand Rapids, MI, or equivalent). The color calibration software constructs an International Color Consortium (ICC) color profile for the scanner, which is used to color correct the output images. The color corrected images are then converted into the CIE L*a*b* color space for subsequent color analysis (a suitable image color analysis software is ImageJ v. 1.52 or equivalent, National Institute of Health, USA).

The absorbent article samples are conditioned at about 23° C.±2° C. and about 50%±2% relative humidity for 2 hours prior to testing. The front and rear panels are separated at the side/hip regions such that the article can be laid flat.

Now secure the front panel to a horizontally flat surface in its fully extended state such that the outward facing surface of the specimen is facing up. With the panel in its fully extended state, pre-strained elastic strands in the panel will be under tension, in an extended state, while nonwoven web material components of the panel, are fully extended laterally and longitudinally but not strained beyond their non-gathered dimensions. Thus, the nonwoven web materials of the specimen are taut but not substantially strained. Determine the side length of an individual square grid unit of the Rugosity Grid by measuring the average strand spacing, as described herein, then multiply this value by 2 and record as the front grid square unit side length to the nearest 0.01 mm. In like fashion, repeat the entire procedure for the rear panel of the article, and record as the rear grid square unit side length to the nearest 0.01 mm.

The scanner is turned on 30 minutes prior to calibration and image acquisition. Deselect any automatic color correction or color management options that may be included in the scanner software. If the automatic color management cannot be disabled for the particular scanner chosen, that scanner is not appropriate for this method. The recommended procedures of the color calibration software are followed to create and export an ICC color profile for the scanner. The color calibration software compares an acquired IT8 target image to a corresponding reference file to create and export the ICC color profile for a scanner, which will be applied within the image analysis program to correct the color of subsequent output images.

Collect images that represent the entirety of both the front and rear panels of the absorbent article sample where the Rugosity Area, as described herein, is present as follows. The scanner lid is opened and the first portion of the front panel is secured in its fully extended state, as previously described, such that the outward facing surface of the panel is facing the scanner glass. Now place a standard white tile, such as that available from Hunter Associates Laboratory, Inc, Reston, VA, or equivalent, behind the secured first portion of the front panel to provide a backing of uniform white color. The lid of the scanner is now closed. A scan of the panel including the printed areas and any surrounding areas is acquired and imported into the image analysis software at 24 bit color with a resolution of 72 dpi (approximately 2.83 pixels per mm) in reflectance mode. The overall dimensions of the scanned area are determined by the analyst, however the sum of all scanned areas must represent the entire region of the front panel where a. Rugosity Area is present. Now repeat the entire procedure for additional portions of the front panel until images of the entire Rugosity Area are collected. In like fashion, collect images of the entire Rugosity Area on the rear panel of the absorbent article. The ICC color profile is assigned to each image producing color corrected sRGB images. Each calibrated image is saved in an uncompressed format to retain the calibrated R,G,B color values, such as a TIFF file, prior to analysis.

Each of the sRGB color calibrated images are individually opened in the color analysis software, and converted into the CIE L*a*b* color space. This is accomplished by the following procedure. First, the sRGB data is scaled into a range of [0, 1] by dividing each of the values by 255. Then the companded sRGB channels (denoted with upper case (R,G,B), or generically V) are linearized (denoted with lower case (r,g,b), or generically v) as the following operation is performed on all three channels (R, G, and B):

$$\\ V ∈ \{ R , G , B \}$$

$$\\ v ∈ \{ r , g , b \}$$

$$\\ v = \{ \begin{matrix}\frac{V}{12.92} if V \leq 0.04045 \\ {( \frac{V + 0.055}{1.055} )}^{2.4} otherwise\end{matrix} \}$$

The linear r, g, and b values are then multiplied by a matrix to obtain the XYZ Tristimulus values according to the following formula:

$$\\ [ \begin{matrix}X \\ Y \\ Z\end{matrix} ] = [ \begin{matrix}0.4124 & 0.3576 & 0.1805 \\ 0.2126 & 0.7152 & 0.0722 \\ 0.0193 & 0.1192 & 0.9505\end{matrix} ] [ \begin{matrix}r \\ g \\ b\end{matrix} ]$$

The XYZ Tristimulus values are rescaled by multiplying the values by 100, and then converted into CIE 1976 L*a*b* values as defined in CIE 15:2004 section 8.2.1.1 using D65 reference white.

The printed pattern and surrounding areas included in each of the CIE L*a*b* images are analyzed as follows. A Rugosity Grid is assigned to each image obtained from the front panel of the absorbent article sample such that the side length of each square unit of surface area of the grid is equal to the previously determined front grid square unit side length. Within each square unit, there exists another grid of pixels in the scan image. Thus, the side length of the individual square unit within the Rugosity Grid that will be analyzed will be rounded such that the side length contains as many whole pixels as possible, and will not contain any partial pixels. The L* a* b* values are measured for each pixel within one square grid unit, and recorded to the nearest 0.01 unit. The ΔE between each pixel and all of the other pixels present within one square grid unit is calculated and recorded to the nearest 0.01 unit using the following equation.

ΔE=\sqrt{(L₂*−L₁* )²+(a₂*−a₁*)²+(b₂*−b₁*)²}

Prepare a cumulative curve of the ΔE distribution histogram versus the range of ΔE values obtained for the square grid unit. Determine the ΔE value closest to the 95 th percentile and report to the nearest 0.01 unit. In like fashion, measure the L* a* b* measurements and ΔE calculations for every square grid unit present on the front panel of the absorbent article sample, reporting the ΔE value closest to the 95th percentile for each. Repeat the entire procedure for the rear panel of the absorbent article sample.

Sum the number of individual grid units present in the Rugosity Area on the front and rear panels of the absorbent article sample and record as Total Grid Units. Now sum the number of individual grid units present on the front and rear panels of the absorbent article sample where the reported ΔE closest to the 95th percentile was at least 4, and record as Color Contrast Grid Units. Calculate the percent of the Rugosity Area where there is a color contrast present by dividing the Color Contrast Grid Units by the Total Grid Units, then multiply by 100 and report as Percent Rugosity Area with a Color Contrast to the nearest 1 percent.

Pant Waist Size Measurement

For purposes herein, a pant waist size is the total straight-line length of the waist edge of the pant, including the waist edges of the front and rear panels, between and inside of any side seams, measured with the front and rear panels in their relaxed conditions, with pre-strained elastic strands in their contracted condition, with any wrinkles or folds (but not gathers created by contraction of the elastic strands) smoothed out. Any suitable measurement tool (such as a ruler) may be used to make the measurement.

The dimensions and values disclosed herein are not to be understood as being strictly limited to the exact numerical values recited. Instead, unless otherwise specified, each such dimension is intended to mean both the recited value and a functionally equivalent range surrounding that value.

Every document cited herein, including any cross referenced or related patent or application, is hereby incorporated herein by reference in its entirety to the extent not inconsistent herewith, unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An adult disposable absorbent pant, comprising:
front and rear panels each comprising stretch laminate material, the stretch laminate material comprising an inner layer and an outer layer, each of the inner and outer layers being formed of nonwoven web material, and a plurality of longitudinally spaced, laterally extending and laterally pre-strained elastic strands disposed between the inner layer and the outer layer,
wherein the front and rear panels are joined to each other along left and right side seams each having a top end and a bottom end; and
an absorbent pad assembly comprising:
a front portion terminating at a front end and a rear portion terminating at a rear end, the front portion being joined to the front panel and the rear portion being joined to the rear panel,
a liquid permeable topsheet, a liquid impermeable backsheet, and an absorbent core structure disposed between the topsheet and the backsheet;
wherein the plurality of elastic strands in at least one of the front and rear panels have an average longitudinal strand spacing of 2 mm to 6 mm, and the plurality of elastic strands impart a Rugosity Area to the at least one of the front and rear panels;
wherein the Rugosity Area bears a pattern of printing, visible from outside the pant,
wherein the pattern of printing imparts a Percent Rugosity Area with a Color Contrast of at least 50 percent, for the at least one of the front and rear panels.

2. The absorbent pant of claim 1, wherein the pattern of printing imparts a Percent Rugosity Area with a Color Contrast of at least 65 percent, for the at least one of the front and rear panels.

3. The absorbent pant of claim 1, wherein the pattern of printing imparts a Percent Rugosity Area with a Color Contrast of at least 75 percent, for the at least one of the front and rear panels.

4. The absorbent pant of claim 1, having a pant waist size of at least 35 cm.

5. The absorbent pant of claim 2, having a pant waist size of at least 35 cm.

6. The absorbent pant of claim 3, having a pant waist size of at least 35 cm.
